(19) United States
(12) Patent Application Publication
(10) Pub. No.: US 2026/0026764 A1
(43) Pub. Date: Jan. 29, 2026

(54) RADIOGRAPHIC IMAGE ANALYSIS PROGRAM, RADIOGRAPHIC IMAGE CAPTURING PROGRAM, RADIOGRAPHIC IMAGE ANALYSIS METHOD, RADIOGRAPHIC IMAGE CAPTURING METHOD, RADIOGRAPHIC IMAGE ANALYSIS APPARATUS, RADIOGRAPHIC IMAGE CAPTURING APPARATUS, AND RADIOGRAPHIC IMAGE SYSTEM

(71) Applicant: Konica Minolta, Inc., Tokyo (JP)

(72) Inventors: Kojiro ONO, Chiba (JP); Naoki HAYASHI, Tokyo (JP)

(21) Appl. No.: 19/278,038

(22) Filed: Jul. 23, 2025

(30) Foreign Application Priority Data
Jul. 29, 2024 (JP) 2024-122327

(51) Int. Cl. A61B 6/00; A61B 6/50; G06T 7/00; G06T 7/246; G06T 11/00

(52) U.S. Cl. CPC A61B 6/486; A61B 6/50; A61B 6/507; A61B 6/5205; G06T 7/0016; G06T 7/248; G06T 11/005; G06T 2207/10116; G06T 2207/30061; G06T 2207/30104

(57) ABSTRACT

The present disclosure provides a non-transitory computer-readable recording medium storing a radiographic image analysis program that causes a computer to execute: acquiring a plurality of frame images generated by performing dynamic imaging by irradiating a subject with radiation under an imaging condition corresponding to a combination of a plurality of types of dynamic analyses set in advance; and executing, based on the plurality of frame images, the plurality of types of dynamic analyses included in the combination.

### CROSS REFERENCE TO RELATED APPLICATIONS

The entire disclosure of Japanese Patent Application No. 2024-122327, filed on Jul. 29, 2024, is incorporated herein by reference in its entirety.

### BACKGROUND

### Technological Field

The present disclosure relates to a non-transitory computer-readable recording medium storing a radiographic image analysis program, a non-transitory computer-readable recording medium storing a radiographic image capturing program, a radiographic image analysis method, a radiographic image capturing method, a radiographic image analysis apparatus, a radiographic image capturing apparatus, and a radiographic image system.

### Description of Related Art

In the related art, a still image capturing technology for capturing an image of radiation that has passed through an inspection target site in a subject with the use of a film, a screen, a photostimulable phosphor plate, or the like, and a radiation diagnostic technology using the still image capturing technology have been widespread. In recent years, on the other hand, a radiation dynamic imaging technology has been developed in which an inspection target site is continuously irradiated with radiation, the transmitted radiation is imaged a plurality of times per unit time using a semiconductor image sensor such as a flat panel detector (FPD), and a moving image of the inspection target site is generated.

For example, Japanese Patent Application Laid-Open No. 2017-205187 discloses a dynamic analysis system in which a lung field region is extracted from a dynamic image of the chest, a feature amount related to the extracted lung-field region is calculated, and, among a series of frame images of the dynamic image, an effective section effective for a dynamic analysis is selected based on the calculated feature amount.

### SUMMARY

There are various types of analyses of a dynamic image (dynamic analysis) depending on a target site to be analyzed. For example, the dynamic analysis of the chest includes extraction of a lung field region, tracking of displacement of the diaphragm, extraction of the tracheal wall, visualization of lung tissue behavior associated with respiration, visualization of blood flow in the lung field synchronized with heartbeat, and the like.

In order to diagnose a specific disease, it is known to be effective to execute a combination of a plurality of types of dynamic analyses in dynamic imaging. For example, for diagnosis of chronic obstructive pulmonary disease (COPD), extraction of a lung field region, extraction of the tracheal wall, visualization of lung tissue behavior associated with respiration, and the like are useful.

Dynamic imaging in which a dynamic image required for a dynamic analysis is captured needs to be performed under imaging conditions corresponding to the type of required dynamic analysis. This is because the body state of the subject at the time of dynamic imaging and the parameters (frame rate, dose, and the like) of frame images required for the dynamic analysis may vary depending on the type of dynamic analysis.

An object of the present disclosure is to provide a non-transitory computer-readable recording medium storing a radiographic image analysis program, a non-transitory computer-readable recording medium storing a radiographic image capturing program, a radiographic image analysis method, a radiographic image capturing method, a radiographic image analysis apparatus, a radiographic image capturing apparatus, and a radiographic image system, each of which is capable of performing a combination of a plurality of dynamic analyses on a subject.

To achieve at least one of the abovementioned objects, according to an aspect of the present invention, a radiographic image analysis program according to an aspect of the present disclosure causes a computer to execute: acquiring a plurality of frame images generated by performing dynamic imaging by irradiating a subject with radiation under an imaging condition corresponding to a combination of a plurality of types of dynamic analyses set in advance; and executing, based on the plurality of frame images, the plurality of types of dynamic analyses included in the combination.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a connection diagram illustrating a system configuration in an embodiment of the present disclosure;

FIG. 2 is a block diagram for describing a functional configuration of an imaging control apparatus;

FIG. 3 is a diagram illustrating an exemplary configuration of a radiographic imaging control apparatus;

FIG. 4 is a diagram illustrating an exemplary configuration of a radiographic image analysis apparatus;

FIG. 5 is a flowchart illustrating an operation example of a radiographic image processing system;

FIG. 6 is a diagram illustrating correspondence relationships between various diseases in the chest and dynamic analyses effective for diagnosing the diseases; and

FIG. 7 is a conceptual diagram illustrating the relationship between the elapsed time and the respiratory depth in a case where dynamic imaging is continuously performed in three respiratory states of eupnea, breath holding, and deep respiration.

### DETAILED DESCRIPTION OF EMBODIMENTS

Hereinafter, one or more embodiments of the present invention will be described with reference to the drawings. However, the scope of the invention is not limited to the disclosed embodiments.

### <Overview>

The present disclosure relates to a radiographic image processing system including a radiographic image capturing apparatus, a radiographic imaging control apparatus, and a radiographic image analysis apparatus.

The radiographic image processing system according to the present disclosure generates a dynamic image by capturing radiographic image data of a subject with the radiographic image capturing apparatus, and performs various types of analysis processing (dynamic analyses) using the radiographic image data in the radiographic image analysis apparatus. The radiographic imaging control apparatus controls imaging processing (dynamic imaging) in the radiographic image capturing apparatus.

Note that, in the present specification, the dynamic imaging refers to acquiring a plurality of images indicating the dynamics of a subject by repeatedly irradiating the subject with radiation, such as X-rays, as pulsed radiation at predetermined time intervals (pulse irradiation) or by continuously irradiating the subject with radiation at a low dose rate without interruption (continuous irradiation). In the present specification, a series of images obtained by dynamic imaging will be referred to as a dynamic image, and each of a plurality of images constituting a dynamic image will be referred to as a frame image. Further, in the present specification, various types of analysis processing performed using a dynamic image will be referred to as a dynamic analysis.

In the radiographic image processing system according to the present disclosure, a combination of a plurality of types of dynamic analyses that is performed on the subject is set before dynamic imaging. A combination of a plurality of types of dynamic analyses may be determined, for example, by a medical doctor who has examined a subject, or the like, based on a disease from which the subject is predicted to suffer.

In the radiographic image capturing apparatus, the generation of a dynamic image (that is, dynamic imaging) is performed based on imaging conditions set according to a combination of a plurality of types of dynamic analyses to be performed on the subject.

By such an operation, the radiographic image processing system of the present disclosure makes it possible to generate radiographic image data required for a combination of a plurality of types of dynamic analyses useful for a specific disease, by one imaging processing, such that a burden on the subject is reduced as much as possible. Accordingly, it is possible to execute a dynamic analysis which is optimal for the subject and has high accuracy, while suppressing a burden on the subject or a user (a medical doctor, an imaging technician, or the like) of the radiation image processing system.

Hereinafter, an embodiment of the present disclosure will be described in detail with reference to the accompanying drawings.

### <System Configuration>

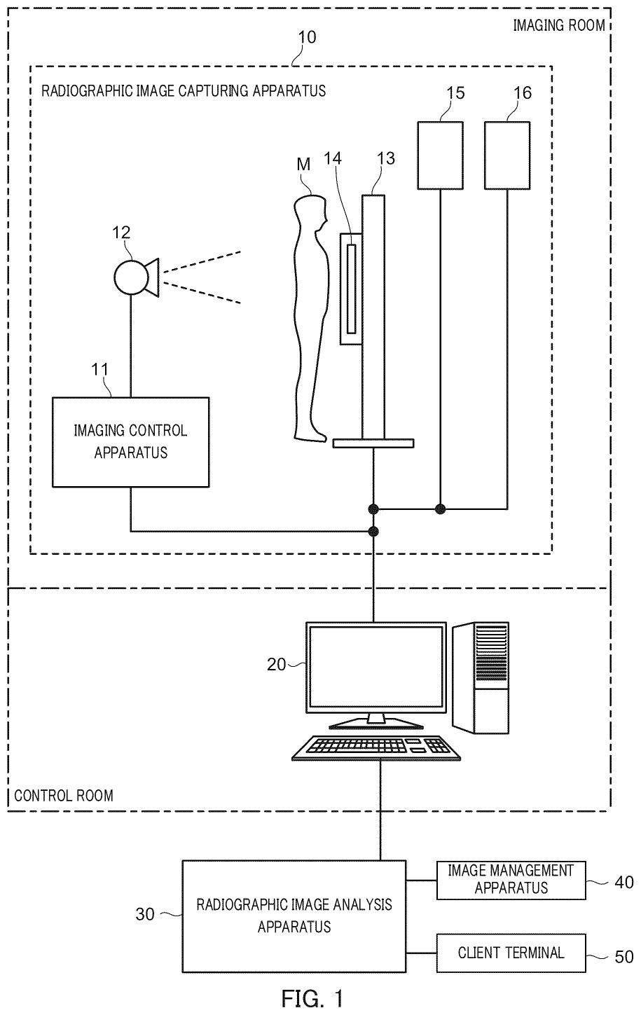

FIG. 1 is a connection diagram illustrating a system configuration in an embodiment of the present disclosure. A radiographic image processing system 100 according to the embodiment of the present disclosure includes a radiographic image capturing apparatus 10, a radiographic imaging control apparatus (console apparatus) 20, a radiographic image analysis apparatus 30, an image management apparatus 40, and a client terminal 50.

In the example illustrated in FIG. 1, the radiographic image capturing apparatus 10 is disposed in an imaging room, and the radiographic imaging control apparatus 20 is disposed in a control room. The radiographic image capturing apparatus 10, the radiographic imaging control apparatus 20, the radiographic image analysis apparatus 30, the image management apparatus 40, and the client terminal 50 are connected to each other via a communication network compliant with, for example, the Digital Image and Communications in Medicine (DICOM) standard or the like.

The radiographic image capturing apparatus 10 performs dynamic imaging based on the control of the radiographic imaging control apparatus 20. A dynamic image generated by the radiographic image capturing apparatus 10 is transmitted to the radiographic image analysis apparatus 30 via the radiographic imaging control apparatus 20. The radiographic image analysis apparatus 30 executes a dynamic analysis based on the dynamic image. A dynamic image and a dynamic analysis result are transmitted to the client terminal 50 and are viewed by a medical worker such as a medical doctor. The dynamic image and the dynamic analysis result are transmitted to and managed by the image management apparatus 40 (for example, a picture archiving and communication system (PACS)).

Each of the radiographic image capturing apparatus 10, the radiographic imaging control apparatus 20, and the radiographic image analysis apparatus 30 is a kind of computer. The computer includes a processor and a memory, and can implement a predetermined function by reading, developing, and executing a program stored in the memory.

### [Radiographic Image Capturing Apparatus 10]

As illustrated in FIG. 1, the radiographic image capturing apparatus 10 includes an imaging control apparatus 11, a radiation irradiation apparatus 12, an imaging table 13, a radiation detector 14, a displaying apparatus 15, and a sound apparatus 16.

The imaging control apparatus 11 acquires setting information on settings of radiation dynamic imaging from the radiation imaging control apparatus 20, sets imaging conditions for performing dynamic imaging based on the setting information, and controls the radiation irradiation apparatus 12 based on the imaging conditions to cause a subject to be irradiated with radiation and imaging to be performed. The imaging control apparatus 11 is constituted by a central processing unit (CPU), a random access memory (RAM), and the like.

The setting information is information on settings for performing dynamic imaging on the subject, and is information on a combination of a plurality of types of dynamic analyses that is performed on the subject by the radiographic image analysis apparatus 30. The setting information is set by the operational person of the radiographic image processing system 100, for example, an imaging technician or the like, in the radiographic imaging control apparatus 20 to be described later.

The imaging conditions include, for example, various conditions such as a pulse rate, a pulse width, a pulse interval, the number of imaging frames per imaging, a dose per unit time of radiation irradiation, and the body state (the respiratory state or the like) of the subject. The pulse rate is the number of times of radiation irradiation per second, and coincides with the frame rate of image data. The pulse width is a radiation irradiation time per radiation irradiation. The pulse interval is a time from the start of one radiation irradiation to the start of the next radiation irradiation, and coincides with a time interval (frame interval) between a plurality of image data. The imaging conditions may be automatically determined by the imaging control apparatus 11 of the radiographic image capturing apparatus 10 based on the setting information.

The radiation irradiation apparatus 12 is disposed at a position facing the radiation detector 14 fixed to the imaging table 13. The radiation irradiation apparatus 12 irradiates radiation (X-rays) according to the control of the imaging control apparatus 11.

The radiation detector 14 is constituted by a semiconductor image sensor such as a flat panel detector (FPD). The radiation detector 14 includes a board in which a plurality of detection elements (pixels) that detects the radiation irradiated by the radiation irradiation apparatus 12 according to the intensity of the radiation, converts the detected radiation into an electrical signal, and accumulates the electrical signal is arranged in a matrix. Each pixel of the board is configured to include a switcher such as a thin film transistor (TFT).

The radiation detector 14 controls the switcher of each pixel based on an image reading conditions inputted through the radiographic imaging control apparatus 20 to read electrical signals accumulated in each pixel, and outputs intensity information for each pixel to an image generator 113. The image reading conditions are, for example, a frame rate, a frame interval, a pixel size, an image size (matrix size), and the like. The frame rate is the number of frame images acquired per second, and coincides with the pulse rate. The frame interval is a time from the start of one operation of acquiring image data to the start of the operation of acquiring the next frame image, and coincides with the pulse interval.

The imaging control apparatus 11 and the radiation detector 14 are connected to each other, and are configured to exchange synchronization signals with each other to synchronize the radiation irradiation operation with the image reading operation.

As described above, the radiographic image capturing apparatus 10 performs dynamic imaging of a radiographic image by the radiation irradiation apparatus 12 irradiating radiation and the radiation detector 14 generating image data based on the intensity of the irradiated radiation under the control of the imaging control apparatus 11.

The displaying apparatus 15 and the sound apparatus 16 provide a subject M with instructions on a posture to be taken and the body state (the respiratory state or the like) when dynamic imaging of the subject M is performed. The displaying apparatus 15 is, for example, a display apparatus such as a cathode ray tube (CRT), a liquid crystal display, or an organic electro luminescence (EL) display. The sound apparatus 16 is, for example, a sound output apparatus such as a speaker. Each of the displaying apparatus 15 and the sound apparatus 16 may provide instructions having the same content to the subject, or only one of the displaying apparatus 15 and the sound apparatus 16 may provide an instruction to the subject.

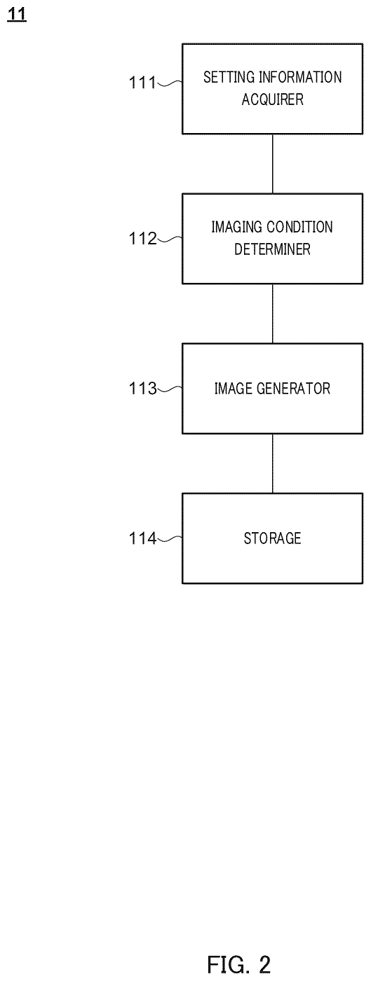

FIG. 2 is a block diagram for describing a functional configuration of the imaging control apparatus 11. The imaging control apparatus 11 includes a setting information acquirer 111, an imaging condition determiner 112, an image generator 113, and a storage 114.

The setting information acquirer 111 acquires setting information from the radiographic imaging control apparatus 20.

The imaging condition determiner 112 determines, based on the setting information, imaging conditions when dynamic imaging of the subject is performed. Information indicating a correspondence relationship(s) between a combination of a plurality of types of dynamic analyses and an imaging condition(s) suitable for the combination, which is indicated by the setting information, is stored in the storage 114 in advance. The imaging condition determiner 112 may determine the imaging conditions by reading the information indicating the correspondence relationship(s) from the storage 114 and collating the information with the setting information.

The image generator 113 executes dynamic imaging of the subject based on the determined imaging condition(s) and generates a radiographic image with a plurality of frames. Specifically, the image generator 113 controls the operations of the radiation irradiation apparatus 12 and the radiation detector 14 based on the imaging condition(s), and generates image data by acquiring, for each pixel, intensity information on the intensity of radiation transmitted through the subject from the radiation detector 14.

As described above, the storage 114 stores, in advance, information indicating a correspondence relationship(s) between a combination of a plurality of types of dynamic analyses and an imaging condition(s) suitable for the combination, and the like.

Details of the operations of the setting information acquirer 111, the imaging condition determiner 112, and the image generator 113, which are included in the imaging control apparatus 11, will be described later.

### [Radiographic Imaging Control Apparatus 20]

The radiographic imaging control apparatus 20 is, for example, a computer such as a personal computer (PC) or a workstation. The radiographic imaging control apparatus 20 may be a desktop computer as in the example illustrated in FIG. 1, or may be a portable computer, that is, a so-called notebook computer or a so-called tablet computer.

The radiographic imaging control apparatus 20 controls dynamic imaging of the radiographic image capturing apparatus 10 by receiving imaging order information from a radiology information system (RIS) or the like and transmitting the imaging order information to the radiographic image capturing apparatus 10. The imaging order information includes various information on dynamic imaging to be executed next, such as patient information, inspection information, the type of analysis, and data attributes (for example, information indicating a site, a body posture, and the like, information on a combination of a plurality of types of dynamic imaging, and the like). The inspection information includes an inspection ID and an inspection target site (for example, the chest, in particular, the lungs, the heart or the like). The types of analysis include, for example, ventilation analysis, pulmonary blood flow analysis, measurement of the maximal ventilation volume, and the like. The data attributes include urgent, general outpatient, ward follow-up, and the like. The imaging order information is generated, for example, when a medical doctor or the like requests the radiographic image processing system 100 to perform dynamic imaging of the subject, or the like.

In addition, the radiographic imaging control apparatus 20 generates setting information indicating at least one combination of a plurality of types of dynamic analyses executable by the radiographic image analysis apparatus 30 based on imaging order information or an input by the operational person.

In a case where the radiographic imaging control apparatus 20 generates the setting information based on the imaging order information, the following sequence can be considered. Each of combinations of numerous dynamic analyses is defined as a preset on the RIS in advance. In this case, for example, a medical doctor selects a combination preset on the RIS according to the type of dynamic analysis image that the medical doctor wants to obtain. Imaging order information including information on the combination selected by the medical doctor is generated by the RIS, and the generated imaging order information is received by the radiographic imaging control apparatus 20. The radiographic imaging control apparatus 20 generates setting information including information indicating the combination of types of dynamic imaging to be performed based on the imaging order information, and transmits the setting information to the imaging control apparatus 11. Accordingly, the radiographic imaging control apparatus 20 can control the dynamic imaging of the radiographic image capturing apparatus 10.

In a case where the radiographic imaging control apparatus 20 generates the setting information based on the imaging order information, the radiographic image capturing apparatus 10 may have in advance information on what types of dynamic imaging in combination can be instructed by the setting information.

In a case where the radiographic imaging control apparatus 20 generates the setting information based on an input by the operational person, the following sequence is considered. The operational person acquires, from the medical doctor who has transmitted the imaging order information on the RIS, information on what type(s) of dynamic imaging the medical doctor desires, by another means (for example, e-mail, telephone, chat, checking a description in an electronic medical record, or the like). The operational person determines, based on the acquired information, which combination is to be performed, and generates setting information including information indicating the combination of the types of dynamic imaging to be performed in the radiographic imaging control apparatus 20. In this case, the radiographic imaging control apparatus 20 and the radiographic image capturing apparatus 10 may have in advance information on what types of dynamic imaging in combination can be set to the setting information. Alternatively, instead of determining what combination is to be performed by the operational person, the radiographic imaging control apparatus 20 may determine what combination of dynamic imaging is to be performed based on various information inputted by the operational person.

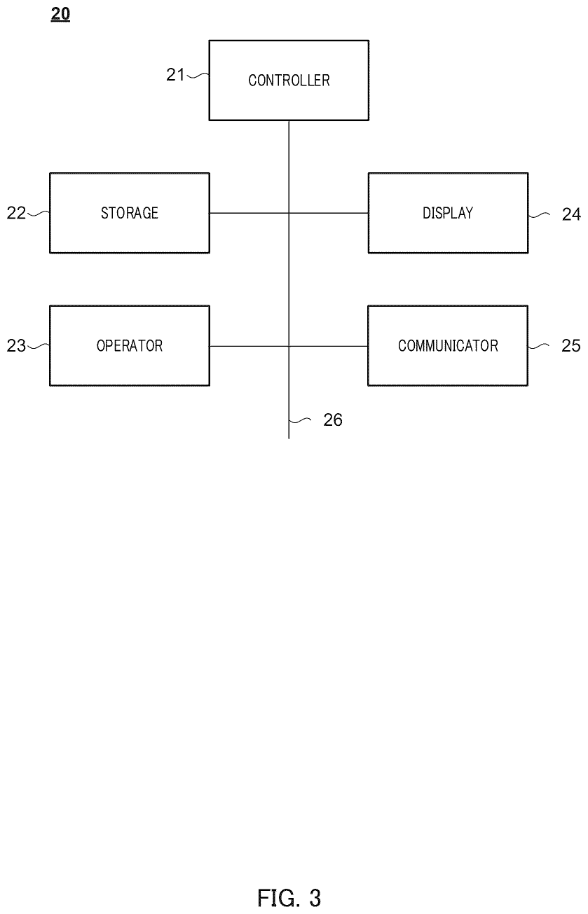

FIG. 3 is a diagram illustrating an example of a configuration of the radiographic imaging control apparatus 20. The radiographic imaging control apparatus 20 includes a controller 21, a storage 22, an operator 23, a display 24, and a communicator 25. The respective configurations included in the radiographic imaging control apparatus 20 are connected to each other by a bus 26.

The radiographic imaging control apparatus 20 outputs, to the radiographic image capturing apparatus 10, setting conditions set by the operational person or the like and imaging order information acquired in advance from the RIS or the like, and controls imaging processing by the radiographic image capturing apparatus 10. The radiographic imaging control apparatus 20 may display a dynamic image generated by the radiographic image capturing apparatus 10, for example, the operational person to check the dynamic image.

The controller 21 is constituted by a CPU, a RAM, and the like. In the controller 21, in response to an operation of the operator 23, the CPU reads a system program and various processing programs stored in the storage 22, develops the system program and various processing programs in the RAM, and controls the operation of each unit of the radiographic imaging control apparatus 20 based on the developed programs.

The storage 22 is constituted by a non-volatile semiconductor memory, a hard disk, or the like. The storage 22 stores various programs to be executed by the controller 21, parameters required for execution of processing by the programs, or data such as processing results. The various programs are stored in the form of readable program codes, and the controller 21 sequentially executes operations according to the program codes.

Further, the storage 22 stores image reading conditions for performing dynamic imaging. Further, the storage 22 stores imaging order information transmitted from the RIS or the like. When the radiographic imaging control apparatus 20 controls the dynamic imaging of the radiographic image capturing apparatus 10, the radiographic imaging control apparatus 20 reads the image reading conditions and the imaging order information corresponding to the subject from the storage 22 and transmits the image reading conditions and the imaging order information.

The operator 23 is an operation device such as a keyboard including a cursor key, number input keys, various function keys and the like, a pointing device such as a mouse or a trackball, a touch screen, and the like. The operator 23 generates an instruction signal based on an input by the operational person, and outputs the instruction signal to the controller 21.

The display 24 is constituted by a displaying device such as a CRT, a liquid crystal display, or an organic EL display. The display 24 displays an input instruction from the operator 23, image data generated by the radiographic image capturing apparatus 10, and the like according to an instruction of a display signal inputted from the controller 21.

The communicator 25 transmits and receives data to and from the radiographic image capturing apparatus 10, the radiographic image analysis apparatus 30, the RIS, and the like.

### [Radiographic Image Analysis Apparatus 30]

The radiographic image analysis apparatus 30 is, for example, a computer such as a PC or a workstation. The radiographic image analysis apparatus 30 may be a desktop computer or a portable computer, that is, a so-called notebook computer or a so-called tablet computer.

The radiographic image analysis apparatus 30 executes, based on the dynamic image captured by the radiographic image capturing apparatus 10 based on the setting information (information on a combination of a plurality of types of dynamic analyses) set in the radiographic imaging control apparatus 20, the plurality of types of dynamic analyses.

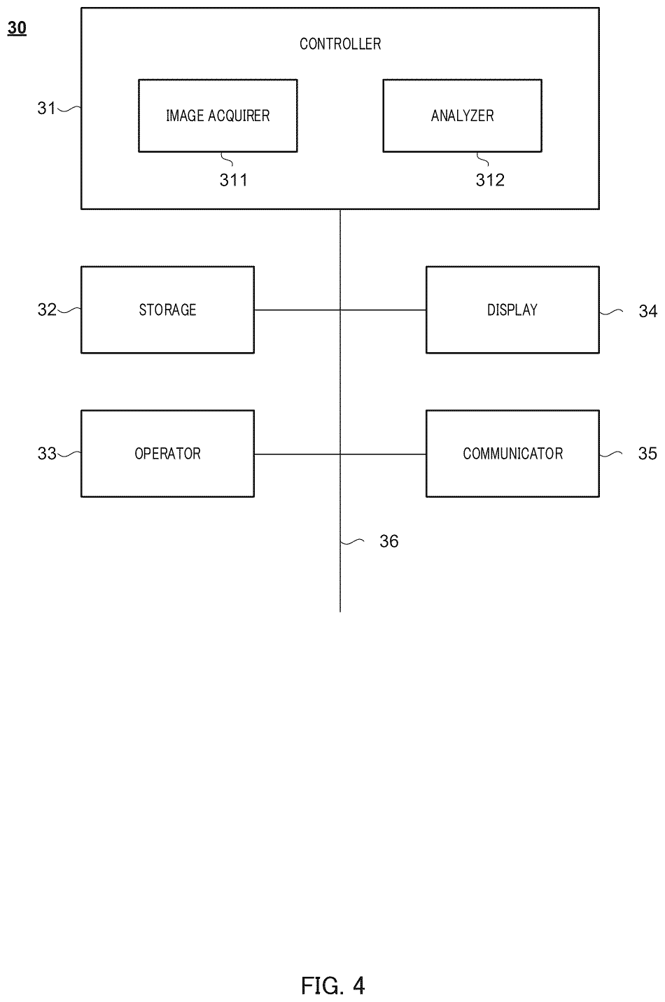

FIG. 4 is a diagram illustrating an exemplary configuration of the radiographic image analysis apparatus 30. The radiographic image analysis apparatus 30 includes a controller 31, a storage 32, an operator 33, a display 34, and a communicator 35. The respective configurations included in the radiographic image analysis apparatus 30 are connected to each other by a bus 36.

The controller 31 is constituted by a CPU, a RAM, and the like. In the controller 31, in response to an operation of the operator 33, the CPU reads a system program and various processing programs stored in the storage 32, develops the system program and various processing programs in the RAM, and executes operation control of each unit of the radiographic image analysis apparatus 30, a dynamic analysis, and the like based on the developed programs.

The controller 31 includes an image acquirer 311 and an analyzer 312. The image acquirer 311 acquires a radiographic image with a plurality of frames generated by the radiographic image capturing apparatus 10. The analyzer 312 executes a plurality of types of dynamic analyses included in a combination based on radiographic image data of a plurality of frames.

Details of the operations of the image acquirer 311 and the analyzer 312 included in the controller 31 will be described later.

The storage 32 is constituted by a non-volatile semiconductor memory, a hard disk, or the like. The storage 32 stores various programs to be executed by the controller 31, parameters required for execution of processing by the programs, or data such as processing results. The various programs are stored in the form of readable program codes, and the controller 31 sequentially executes operations according to the program codes.

The storage 32 also stores patient information, inspection information, and list information indicating the status (for example, the progress status such as during reception, during dynamic analysis, completion of analysis, and the like) according to each dynamic image, which are for generating dynamic images generated by the radiographic image capturing apparatus 10. Further, analysis results are stored in the storage 32 in association with the dynamic images.

The operator 33 is an operation device such as a keyboard including a cursor key, number input keys, and various function keys, a pointing device such as a mouse or a trackball, and a touch screen. The operator 33 generates an instruction signal based on an input by the operational person, and outputs the instruction signal to the controller 31. Further, the operator 33 may include a touch screen on the display screen of the display 34, and in this case, outputs an instruction signal inputted through the touch screen to the controller 31.

The display 34 is constituted by a display device such as a CRT, a liquid crystal display, or an organic EL display. The display 34 displays an input instruction from the operator 33, image data generated by the radiographic image capturing apparatus 10, and the like according to an instruction of a display signal inputted from the controller 31.

The communicator 35 transmits and receives data to and from the radiographic image capturing apparatus 10, the radiographic image analysis apparatus 30, and the like.

### <Operation Example of Entire System>

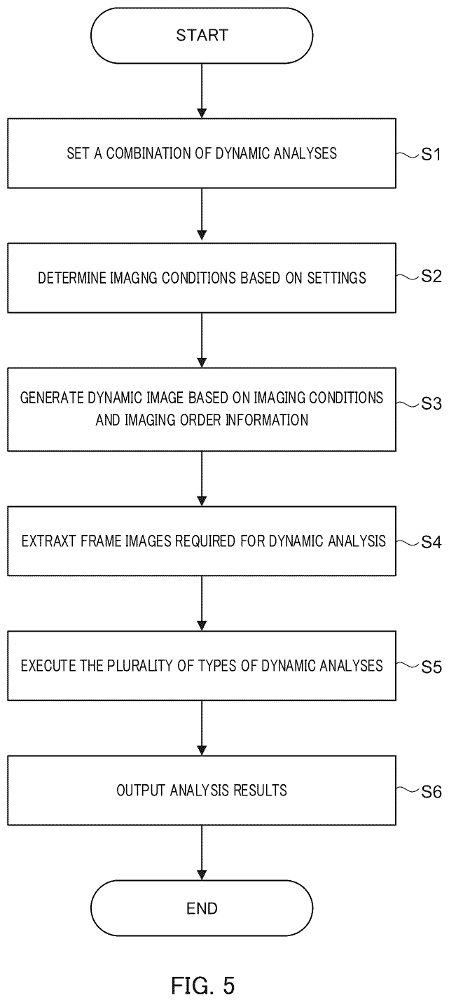

Next, an operation example of the radiographic image processing system 100 in its entirety will be described. FIG. 5 is a flowchart illustrating an operation example of the radiographic image processing system 100.

In step S1, in the radiographic imaging control apparatus 20, a combination of a plurality of dynamic analyses to be executed is set among a plurality of types of dynamic analyses using a dynamic image.

For example, it is assumed that a medical doctor or the like examines a certain subject (patient) and predicts that the subject suffers from a specific disease from the symptom. It is known that the radiographic image processing system 100 can perform an analysis effective for the specific disease by combining and executing some of a plurality of types of executable dynamic analyses.

Alternatively, for example, a disease of a subject transported for emergency may not be able to be predicted in advance. In this case, in the radiographic image processing system 100, a combination of dynamic analyses useful for diagnosis of the subject may be determined in advance among a plurality of types of executable dynamic analyses.

Examples of the types of dynamic analyses in a case where the chest (lungs) of the subject is an imaging target include chest bone attenuation processing, frequency enhancement processing, diaphragm movement tracking processing, lung field movement amount visualization processing, lung field area measurement processing, tracheal diameter measurement processing, ventilation analysis processing, blood flow analysis processing, and second blood flow analysis processing.

The chest bone attenuation processing is processing of attenuating a signal of a rib or a clavicle in the lung field. The chest bone attenuation processing makes it possible to generate a lung field image in which bones are not displayed in an overlapping manner.

The frequency enhancement processing is processing of enhancing the movement of the intra-lung tissue having a specific frequency. The frequency enhancement processing makes it possible to enhance the visibility by making edge portions of the tissue conspicuous.

The diaphragm movement tracking processing is processing of tracking the up/down movement of the lung apex and the diaphragm ridge. The diaphragm movement tracking processing makes it possible to track the movement of the diaphragm and quantify the movement amount in the up-down direction.

The lung field movement amount visualization processing is processing of tracking a signal value pattern including a blood vessel shadow or the like in the lung field and displaying, as a vector, a movement amount of each region associated with respiration by using the standard inhalation level frame as a reference. The lung field movement amount visualization processing makes it possible to visualize movement in the lung field.

The lung field area measurement processing is processing of extracting and determining a lung field contour and measuring a lung field area.

The tracheal diameter measurement processing is processing of extracting a tracheal wall and measuring a change in the tracheal diameter due to respiration.

The ventilation analysis processing is processing of visualizing a signal change in the behavior of lung tissue associated with respiration. The ventilation analysis processing makes it possible to visualize the respiratory state of the patient.

The blood flow analysis processing is processing of visualizing a signal change in the lung field synchronized with the heartbeat. The blood flow analysis processing makes it possible to visualize the movement of the blood flow in the lung field.

The second blood flow analysis processing is processing of visualizing the amount of change in the high-frequency signal in the lung field synchronized with the heartbeat. According to the second blood flow analysis processing, it is possible to express a smaller change in the amount of blood flow than in the blood flow analysis processing.

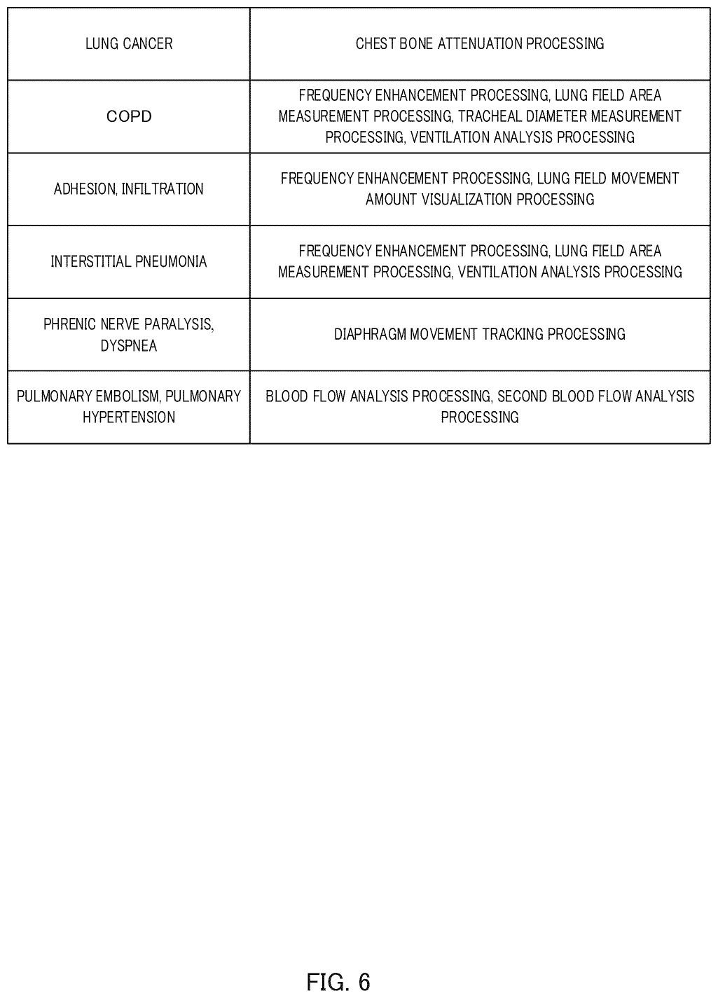

FIG. 6 is a diagram illustrating correspondence relationships between various diseases in the chest and dynamic analyses effective for diagnosing the diseases. In a case where the subject suffers from lung cancer, it is known that the analysis by the chest bone attenuation processing is effective. In a case where the subject suffers from chronic obstructive pulmonary disease (COPD), it is known that the analysis by the frequency enhancement processing, the lung field area measurement processing, the tracheal diameter measurement processing, and the ventilation analysis processing is effective. In a case where the subject suffers from adhesion or infiltration, it is known that the analysis by the frequency enhancement processing and the lung field movement amount visualization processing is effective. In a case where the subject suffers from interstitial pneumonia, it is known that analysis by the frequency enhancement processing, the lung field area measurement processing, and the ventilation analysis processing is effective. In a case where the subject suffers from phrenic nerve paralysis or dyspnea, it is known that the analysis by the diaphragm movement tracking processing is effective. In a case where the subject suffers from pulmonary embolism or pulmonary hypertension, it is known that the analysis by the blood flow analysis processing and the second blood flow analysis processing is effective. Thus, the type(s) of effective dynamic analysis/analyses and a combination of effective dynamic analyses vary depending on the disease to be predicted.

Further, in a case where the disease of the subject cannot be predicted in advance, for example, the type(s) of dynamic analysis/analyses and a combination of dynamic analyses may be set in advance according to the medical doctor who diagnoses the subject. Alternatively, for example, the type(s) of dynamic analysis/analyses and a combination of dynamic analyses may be set in advance according to the facility or the department to which the medical doctor who diagnoses the subject belongs. As a specific example, in a case where the disease of the subject cannot be specified in advance by the medical doctor, the facility, or the department, the type(s) of dynamic analysis/analyses and a combination of dynamic analyses to be performed first are determined in some cases. In such a case, for each medical doctor who diagnoses the subject, facility, or department, the type(s) of dynamic analysis/analyses and a combination of dynamic analyses to be performed may be determined based on the history of the subject up to the dynamic analysis or the like. The history of the subject up to the dynamic analysis includes, for example, a plurality of histories such as whether the subject has been transported for emergency, whether the subject has fever, whether the subject is coughing, and whether the subject has hit his/her head.

The type of dynamic analysis to be performed in the next dynamic imaging is included in the imaging order information. For this reason, in step S1, the operational person performs, on the radiographic imaging control apparatus 20, setting processing of selecting and setting an effective dynamic analysis/analyses among a plurality of types of dynamic analyses executable by the radiographic image processing system 100, based on the imaging order information. The setting processing in step S1 is executed by, for example, the operational person operating the operator 23 of the radiographic imaging control apparatus 20.

For example, in a case where the disease of the subject is predicted to be COPD, the operational person selects a combination of the frequency enhancement processing, the lung field area measurement processing, the tracheal diameter measurement processing, and the ventilation analysis processing by operating the operator 23. On the other hand, for example, in a case where the disease of the subject is predicted to be interstitial pneumonia, the operational person selects a combination of the frequency enhancement processing, the lung field area measurement processing, and the ventilation analysis processing.

The radiographic imaging control apparatus 20 generates, based on the operation of the operational person, setting information indicating the setting of a combination of a plurality of types of dynamic analyses. The radiographic imaging control apparatus 20 transmits the generated setting information and the imaging order information acquired from the RIS or the like in advance to the radiographic image capturing apparatus 10.

In step S2, in the radiographic image capturing apparatus 10, imaging conditions are determined based on the setting information and the imaging order information.

The imaging conditions to be applied at the time of dynamic imaging vary depending on the type of dynamic analysis to be executed. Information on the imaging conditions corresponding to each dynamic analysis is stored in advance in the storage 114 of the radiographic image capturing apparatus 10. In step S2, the setting information acquirer 111 acquires the setting information from the radiographic imaging control apparatus 20, and the imaging condition determiner 112 determines the imaging conditions to be applied to dynamic imaging to be executed next by referring to the storage 114 based on the setting information.

As described above, the imaging conditions include various conditions such as a pulse rate, a pulse width, a pulse interval, the number of imaging frames per imaging, a dose per unit time of radiation irradiation, and the body state of the subject. The body state of the subject means the body state to be taken by the subject at the time of dynamic imaging. In the dynamic imaging of the chest, the body state of the subject includes a respiratory state of the subject, that is, for example, eupnea, breath holding, deep respiration, or the like.

For example, the dynamic analysis in the ventilation analysis processing, one-time tidal volume measurement (tidal volume), or the like requires a dynamic image generated by causing the subject to perform eupnea. The dynamic analysis such as the blood flow analysis processing and the second blood flow analysis processing requires a dynamic image generated by causing the subject to temporarily hold respiration (breath holding). The lung field area measurement processing requires a dynamic image generated by causing the subject to take deep respiration and then temporarily hold respiration (breath holding). The dynamic analysis such as the lung field movement amount visualization processing, the ventilation analysis processing, the diaphragm movement tracking processing, or the tracheal diameter measurement processing requires a dynamic image generated by causing the subject to take deep respiration. However, the above-described dynamic image of the respiratory state is not necessarily required for each dynamic analysis, and the analysis itself is not impossible even when a dynamic image of another respiratory state is used. For example, it is also possible to perform the dynamic analysis of the second blood flow analysis processing by using a dynamic image at the time of deep respiration.

As described above, imaging conditions corresponding to dynamic analyses included in a specific combination among a plurality of types of dynamic analyses may be set in advance with respect to the combination, and imaging may be performed using the imaging conditions set in advance according to the combination at the time of dynamic imaging. The setting of imaging conditions corresponding to dynamic analyses included in a specific combination with respect to the combination may be stored in advance in the storage 114 or the like, for example.

In step S3, a dynamic image of the subject is generated in the radiographic image capturing apparatus 10 based on the determined imaging conditions and the imaging order information.

In step S3, the displaying apparatus 15 and the sound apparatus 16 first check the name of the subject, position the subject, and provide an instruction on the posture to be taken, and the like, based on the imaging conditions and the imaging order information. When sensors included in the radiographic image capturing apparatus 10 detect that the subject takes the posture to be taken and is positioned at an appropriate position, the image generator 113 performs dynamic imaging based on the imaging conditions. A sensor is provided at, for example, a belt for fixing the body of the subject. In a case where the body state of the subject needs to be changed during the dynamic imaging, the displaying apparatus 15 and the sound apparatus 16 instruct the subject to change the direction and posture of the body. The image generator 113 repeats, based on the imaging order information, the instruction and the dynamic imaging until the dynamic imaging to be performed on the subject is completed.

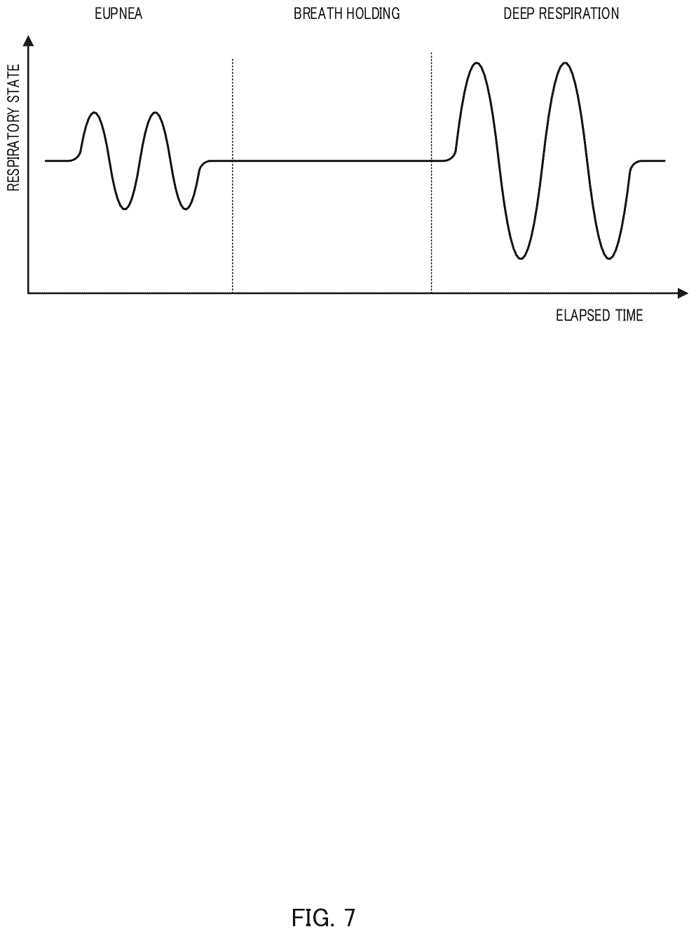

Here, as examples of the body state, operations in step S3 in the case of performing dynamic imaging in three respiratory states of eupnea, breath holding, and deep respiration will be described with specific examples. FIG. 7 is a conceptual diagram illustrating the relationship between the elapsed time and the respiratory depth in a case where dynamic imaging is continuously performed in three respiratory states of eupnea, breath holding, and deep respiration. First, for performing dynamic imaging during eupnea, the displaying apparatus 15 and the sound apparatus 16 instruct the subject to take normal respiration at rest. Then, the image generator 113 performs dynamic imaging based on the imaging conditions corresponding to the eupnea.

In a case where a dynamic image during eupnea can be generated by the image generator 113, the displaying apparatus 15 and the sound apparatus 16 next instruct the subject to hold his/her breath for performing dynamic imaging during breath holding. Here, the displaying apparatus 15 and the sound apparatus 16 may instruct the subject to inhale and then hold his/her breath, or exhale and then hold his/her breath. Then, the image generator 113 performs dynamic imaging based on the imaging conditions corresponding to the time of breath holding.

In a case where a dynamic image during breath holding can be generated by the image generator 113, the displaying apparatus 15 and the sound apparatus 16 next instruct the subject to take deep respiration. Then, the image generator 113 performs dynamic imaging based on the imaging conditions corresponding to the time of breath holding. Note that, although a respiration instruction whereby a burden on the subject becomes less can be given by instructing the respiratory states in the order of eupnea, breath holding, and deep respiration, the respiratory states do not necessarily have to be instructed in the order of eupnea, breath holding, and deep respiration, and the order of instructions may be changed. In addition, for example, in a case where the subject is an elderly person, a COPD patient, or the like, it may be difficult to hold his/her breath, and in such a case, the breath holding may not be instructed. As described above, any one of the respiratory states may not be instructed according to the symptom, the body state, or the like of the subject.

In a case where dynamic imaging needs to be performed under different imaging conditions for each body state, the image generator 113 may perform imaging by changing the pulse rate, the pulse width, the pulse interval, or the like according to each dynamic analysis, for example, while the displaying apparatus 15 and the sound apparatus 16 instruct one respiratory state. For example, in a case where dynamic imaging for the ventilation analysis processing and the diaphragm movement tracking processing is required during deep respiration, the image generator 113 may perform imaging at a pulse rate, a pulse width, and a pulse interval each of which corresponds to the ventilation analysis processing and the diaphragm movement tracking processing, respectively, while the displaying apparatus 15 and the sound apparatus 16 instruct deep respiration.

By such an operation, dynamic imaging for a plurality of types of dynamic analyses can be performed on one subject at a time.

In the above-described example, the aspect in which the imaging conditions are changed for each type of dynamic analysis in one body state has been described. However, for example, the imaging conditions may be determined to some extent for each body state regardless of the type of dynamic analysis. For example, regardless of the type of dynamic analysis, imaging conditions may be set such that dynamic imaging is performed at a relatively low pulse rate and a relatively low dose during eupnea, and imaging conditions may be set such that dynamic imaging is performed at a pulse rate higher than that during eupnea and a relatively high dose during breath holding. In this case, information on the imaging conditions to be set according to the body state of the subject may be stored in the storage 114 in advance.

Further, the same imaging conditions may not be necessarily used in dynamic imaging for one type of dynamic analysis, but imaging conditions for a specific type of dynamic analysis among a plurality of types of dynamic analyses may be varied based on the type of another dynamic analysis included in a combination. In other words, even in the same type of dynamic analysis, the imaging conditions may be changed depending on the type of a combination counterpart.

A specific example will be described. For example, a case where a combination counterpart of the second blood flow analysis processing is the ventilation analysis processing will be considered. The second blood flow analysis processing usually requires a dynamic image during breath holding, and the ventilation analysis processing usually requires a dynamic image during deep respiration. In such a case, the dynamic analysis used in the combination of the second blood flow analysis processing and the ventilation analysis processing may be performed using a dynamic image captured in a state in which the subject is caused to hold his/her breath after taking deep respiration. As described above, it is possible to perform dynamic analysis with high accuracy by performing a plurality of types of dynamic analyses in combination by using a dynamic image captured under imaging conditions satisfying as many imaging conditions required for each dynamic analysis as possible.

On the contrary, a plurality of types of dynamic analyses in combination may be executed using a dynamic image satisfying only the imaging conditions for the dynamic analysis of a combination counterpart. As a specific example, for example, although the second blood flow analysis processing usually requires a dynamic image during breath holding, in a case where a combination counterpart is a dynamic analysis such as the ventilation analysis processing which requires a dynamic image during deep respiration, not a dynamic image during breath holding but a dynamic image during deep respiration may be used as the dynamic image for the second blood flow analysis processing. That is, in a case where a combination includes the second blood flow analysis processing and the ventilation analysis processing, combined dynamic analyses may be performed using an image captured under imaging conditions corresponding to the ventilation analysis processing (an image captured by instructing the subject to take deep respiration at a relatively low pulse rate and a relatively low dose). Accordingly, it is possible to perform a dynamic analysis including the second blood flow analysis processing even on a subject who is difficult to hold his/her breath.

Varying the imaging conditions for a specific type of dynamic analysis based on the type of another dynamic analysis included in a combination is useful, for example, from the viewpoint that the imaging conditions can be appropriately set according to the physical strength, conditions, and the like of the subject. For example, since the act of breath holding imposes a large burden on a relatively physically weak subject (for example, an elderly person or a patient transported for emergency), a dynamic analysis of a type whose imaging conditions include breath holding is unlikely to be performed on a relatively physically weak subject. Accordingly, in a case where a dynamic analysis in which the second blood flow analysis processing and the ventilation analysis processing are combined is performed on a relatively young subject who is relatively physically strong, the analysis can be performed with high accuracy by using a dynamic image captured under imaging conditions satisfying as many imaging conditions for the second blood flow analysis processing and the ventilation analysis processing as possible. On the other hand, in a case where the second blood flow analysis processing is required for the subject who has difficulty in holding his/her breath, it is possible to perform an analysis with less burden on the subject by combining the second blood flow analysis processing with the ventilation analysis processing or the like, in which breath holding is not included in the imaging conditions, and by using a dynamic image captured under the imaging conditions satisfying at least either the imaging conditions for the second blood flow analysis processing or the imaging conditions for the ventilation analysis processing.

Note that, at the time of dynamic imaging, the image generator 113 may generate each generated frame image in association with imaging information indicating, for example, imaging conditions under which the each frame image has been captured. In addition, the image generator 113 may acquire, by using a sensor or the like of the radiographic image capturing apparatus 10, information indicating whether the subject correctly takes the body state to be taken at the time of capturing each frame image, and may associate the information with the each frame image by including the information in imaging information. In this case, for example, the radiographic image capturing apparatus 10 may include a sensor for detecting the respiratory state of the subject on a belt or the like for fixing the body of the subject during imaging, and may detect the respiratory state of the subject during dynamic imaging based on the detection result of the sensor.

In the above-described example, the case has been described in which the displaying apparatus 15 and the sound apparatus 16 automatically provide, to the subject, various instructions regarding the dynamic imaging to be performed, based on the setting conditions and the imaging order information. In the present disclosure, the displaying apparatus 15 and the sound apparatus 16 may not automatically provide various instructions in this manner. For example, the operational person may provide various instructions to the subject verbally or by gesture while causing the setting conditions and the radiographic imaging order information to be displayed on the display 24 or the like of the radiographic imaging control apparatus 20 and checking the content thereof.

The dynamic image generated in step S3 is transmitted from the radiographic image capturing apparatus 10 to the radiographic image analysis apparatus 30 via the radiographic imaging control apparatus 20.

In step S4, in the radiographic image analysis apparatus 30, frame images required for each dynamic analysis to be performed are extracted from the dynamic image received from the radiographic image capturing apparatus 10.

As described above, a dynamic image during eupnea is required for a dynamic analysis during eupnea. A dynamic image during breath holding is required for a dynamic analysis such as the blood flow analysis processing or the second blood flow analysis processing. A dynamic analysis such as the ventilation analysis processing, the diaphragm movement tracking processing, or the tracheal diameter measurement processing requires a dynamic image during deep respiration.

Accordingly, in a case where the blood flow analysis processing is included in the combination set in step S1, frame images corresponding to the time of breath holding are extracted in step S4. In addition, in a case where the ventilation analysis processing is included in the combination set in step S1, frame images corresponding to the time of deep respiration are extracted in step S4. Which frame image is an image corresponding to the time of breath holding or the time of deep respiration may be determined based on the imaging information associated with each frame image.

In addition, frame images having a narrower range may be extracted depending on the type of the dynamic analysis. For example, in the ventilation analysis processing (visualization of lung tissue behavior) and the diaphragm movement tracking processing (quantification of diaphragm displacement), it is theoretically sufficient to have frame images at the time of maximum inhalation and frame images at the time of maximum exhalation in deep respiration, and it can be said that other frame images are not required. Accordingly, in a case where the ventilation analysis processing or the diaphragm movement tracking processing is selected, for example, images of several frames including the maximum inhalation and images of several frames including the maximum exhalation may be extracted from a plurality of frame images captured during deep respiration. Note that, which frame image corresponds to the maximum inhalation and which frame image corresponds to the maximum exhalation among a plurality of frame images captured during deep respiration may be determined based on the imaging information generated during dynamic imaging.

In addition, in step S3, the extraction of frame images used in a specific type of dynamic analysis among a plurality of types of dynamic analyses may be varied based on the type of another dynamic analysis included in a combination.

A specific example will be described. In a case where a combination of the ventilation analysis processing and the diaphragm movement tracking processing is set, the radiographic image analysis apparatus 30 extracts, as frame images to be used in the ventilation analysis processing, images of several frames before and after the maximum inhalation and images of several frames before and after the maximum exhalation. On the other hand, in a case where a combination of the ventilation analysis processing and the tracheal diameter measurement processing is set, the radiographic image analysis apparatus 30 may extract all the frame images generated in a state of deep respiration as the frame images used in the ventilation analysis processing. The reason why all the frame images are extracted with the combination of the ventilation analysis processing and the tracheal diameter measurement processing is that all the frame images in the state of deep respiration are required for the tracheal diameter measurement processing (a mode for measuring the tracheal diameter) which is a combination counterpart. The radiographic image analysis apparatus 30, however, extracts the frame images to be used in the dynamic analysis of a combination counterpart (the diaphragm movement tracking processing or the tracheal diameter measurement processing in the above example) separately from the extraction of the frame images to be used in the ventilation analysis processing. Then, the radiographic image analysis apparatus 30 may perform the analysis by using the same or different frame images for each dynamic analysis included in the combination.

In this way, in step S4, required frame images are extracted for each dynamic analysis included in the combination of the plurality of types of dynamic analyses set in step S1.

In step S5, in the radiographic image analysis apparatus 30, each of the plurality of types of dynamic analyses included in the combination set in step S1 is executed by using the frame images extracted in step S4 for each dynamic analysis. For example, the analyzer 312 of the radiographic image analysis apparatus 30 performs the dynamic analyses, automatically by using a predetermined parameter group, or by using a parameter group or the like set by an operation of the operational person. As specific examples of the dynamic analyses, for example, dynamic analyses known in the art, such as a ventilation analysis and a pulmonary blood flow analysis described in Japanese Patent Application Laid-Open No. 2012-110451 or the like, can be applied.

In step S6, in the radiographic image analysis apparatus 30, the results of the dynamic analyses performed in step S5 are outputted. The output of the analysis results is performed, for example, by displaying the results of the dynamic analyses on the display 34. Alternatively, the output of the analysis results is performed by, for example, transmitting analysis result information indicating the results of the dynamic analyses to a terminal apparatus or the like used by the medical doctor or the like via the communicator 35. Thus, the medical doctor or the like who has requested the dynamic imaging and the dynamic analyses can perform diagnosis or the like based on the analysis results.

Note that, in the operation example illustrated in FIG. 5, the example in which a dynamic image used in each dynamic analysis is captured based on imaging conditions for a plurality of types of dynamic analyses included in a combination has been described. In the present disclosure, for example, a dynamic image may be captured under imaging conditions that can cope with any types of dynamic analyses in combination.

A specific example will be described. It is assumed that a plurality of types of dynamic analyses executable by the radiographic image analysis apparatus 30 uses, for example, at least one of a dynamic image captured when the subject is at rest, a dynamic image captured when the subject is caused to take deep respiration, and a dynamic image captured when the subject is caused to hold his/her breath. In this case, in step S2, the radiographic image capturing apparatus 10 may set imaging conditions such that the imaging conditions include every imaging condition such that any dynamic analysis is executable regardless of the combination set in step S1.

In this case, in step S4, the radiographic image analysis apparatus 30 may respectively extract frame images to be used in each dynamic analysis according to the imaging conditions for dynamic images required for the dynamic analyses included in the combination. Even by such an operation, the radiographic image processing system 100 can perform a plurality of types of dynamic analyses in combination without any problem.

### <Actions and Effects>

As described above, in the radiation image processing system 100 according to the embodiment of the present disclosure, a combination of a plurality of types of dynamic analyses is set before the start of dynamic imaging, the dynamic imaging is performed under imaging conditions corresponding to each dynamic analysis included in the combination based on setting conditions indicating the combination, and the plurality of types of dynamic analyses included in the combination is executed based on a dynamic image generated by the dynamic imaging.

According to the radiographic image processing system 100, such a configuration makes it possible to perform a plurality of types of dynamic analyses as a series of operations, for example, in a case where the plurality of types of dynamic analyses is required for diagnosing a subject. Thus, for example, in a case where a specific disease is estimated based on the symptom of the patient, a plurality of types of dynamic analyses effective for the estimated disease can be performed at once. For this reason, it is possible to reduce the labor required for the operational person or the like of the radiographic image processing system 100 in a case where a plurality of types of dynamic analyses is performed. In addition, since the time for which the subject is restrained for the dynamic imaging can be shortened as compared with a case in which each of a plurality of types of dynamic imaging is separately performed, a burden on the subject can also be reduced.

### <Variations>

Although a preferable example of the present disclosure has been described in the above-described embodiment, the present disclosure is not limited to the above-described embodiment. The present disclosure can be modified in various ways within the scope of the claims.

In the above-described embodiment, as an application example of the radiation image processing system of the present disclosure, the case where a dynamic image of the chest is captured and the dynamic analysis of the chest is executed has been described. The radiographic image processing system according to the present disclosure is not limited thereto, and may perform dynamic imaging and dynamic analysis of another site.

### <Variation 1>

For example, the heart of the subject may be the target of dynamic imaging and dynamic analysis. In this case, the types of dynamic analyses may be different from those for the lungs described in the above-described embodiment. Nonetheless, the second blood flow analysis processing is processing of visualizing the amount of change of a high-frequency signal in the lung field synchronized with the heartbeat, and is therefore executable even in a case where the heart is the subject.

In a case where the heart of the subject is the target of dynamic imaging and dynamic analysis, frame images that do not include arrhythmia (bradycardia, tachycardia, premature contraction, or the like) may be extracted and the dynamic analysis may be performed when frame images required for the dynamic imaging are extracted from a generated dynamic image. A known image analysis technique can be applied to a method of extracting a frame image including arrhythmia.

### <Variation 2>

For example, the throat, the esophagus and the like of the subject may be the targets of dynamic imaging and dynamic analysis. In this case, it is useful for diagnosis of swallowing ability or the like of the subject. As an imaging condition, a state in which the subject swallows a contrast agent or the like may be included as the body state. In this case, when frame images required for dynamic imaging are extracted from a generated dynamic image, frame images in which the contrast agent is passing through the throat and frame images in which the contrast agent is not passing through the throat may be separated and used in different dynamic analyses, respectively. Further, frame images in which the contrast agent is passing through the throat, frame images before the passage, and frame images after the passage may be separately extracted. In this case, it is possible to evaluate whether a matter swallowed once has flowed backward.

Alternatively, a frame image may be extracted each time the contrast agent is passing through the throat. Specifically, frame images in which the contrast agent is passing through the nasopharynx region, frame images in which the contrast agent is passing through the oropharynx region, and frame images in which the contrast agent is passing through the hypopharynx region may be extracted, respectively. Thus, the swallowing ability of the subject can be analyzed with high accuracy.

### <Variation 3>

For example, a joint (elbow, knee, waist, neck, or the like) of the subject may be the target of dynamic imaging and dynamic analysis. In this case, for example, frame images at the time of maximum flexion and frame images at the time of maximum extension may be separately extracted.

Although embodiments of the present invention have been described and illustrated in detail, the disclosed embodiments are made for purposes of illustration and example only and not limitation. The scope of the present invention should be interpreted by terms of the appended claims.

What is claimed is:

1. A non-transitory computer-readable recording medium storing a radiographic image analysis program that causes a computer to execute:
acquiring a plurality of frame images generated by performing dynamic imaging by irradiating a subject with radiation under an imaging condition corresponding to a combination of a plurality of types of dynamic analyses set in advance; and
executing, based on the plurality of frame images, the plurality of types of dynamic analyses included in the combination.

2. The non-transitory computer-readable recording medium storing the radiographic image analysis program according to claim 1, wherein
the executing of the plurality of types of dynamic analyses includes:
extracting, from the plurality of frame images, a frame image that is used in each of the plurality types of dynamic analyses for each of the plurality of types of dynamic analyses included in the combination; and
executing each of the plurality types of dynamic analyses based on the frame image that has been extracted.

3. The non-transitory computer-readable recording medium storing the radiographic image analysis program according to claim 2, wherein
in the extracting of the frame image, the extracting of the frame image that is used in a specific type of dynamic analysis among the plurality of types of dynamic analyses is varied based on a type of another dynamic analysis included in the combination.

4. A non-transitory computer-readable recording medium storing a radiographic image capturing program that causes a computer to execute:
acquiring setting information on a combination of a plurality of types of dynamic analyses;
determining an imaging condition for dynamic imaging based on the setting information; and
generating a plurality of frame images by executing dynamic imaging of a subject by irradiating the subject with radiation based on the imaging condition that has been determined.

5. The non-transitory computer-readable recording medium storing the radiographic image capturing program according to claim 4, wherein
in the determining of the imaging condition, the imaging condition is determined based on the plurality of types of dynamic analyses included in the combination.

6. The non-transitory computer-readable recording medium storing the radiographic image capturing program according to claim 5, wherein
in the determining of the imaging condition, an imaging condition for a specific type of dynamic analysis among the plurality of types of dynamic analyses is varied based on a type of another dynamic analysis included in the combination.

7. A radiographic image analysis method, comprising:
acquiring, by a computer included in an analysis apparatus for a radiographic image, a plurality of frame images generated by performing dynamic imaging by irradiating a subject with radiation under an imaging condition corresponding to a combination of a plurality of types of dynamic analyses set in advance; and
executing, by the computer, based on the plurality of frame images, the plurality of types of dynamic analyses included in the combination.

8. A radiographic image capturing method, comprising:
acquiring, by a computer included in an imaging apparatus for a radiographic image, setting information on a combination of a plurality of types of dynamic analyses using the radiographic image;
determining, based on the setting information, an imaging condition when performing dynamic imaging by irradiating a subject with radiation; and
generating a plurality of frame images by executing the dynamic imaging of the subject based on the imaging condition that has been determined.

9. A radiographic image analysis apparatus, comprising:
a hardware processor configured to:
acquire a plurality of frame images generated by performing dynamic imaging by irradiating a subject with radiation under an imaging condition corresponding to a combination of a plurality of types of dynamic analyses set in advance; and
execute, based on the plurality of frame images, the plurality of types of dynamic analyses included in the combination.

10. A radiographic image capturing apparatus, comprising:
a hardware processor configured to:
acquire setting information on a combination of a plurality of types of dynamic analyses;
determine, based on the setting information, an imaging condition for dynamic imaging; and
generate a plurality of frame images by executing the dynamic imaging of a subject by irradiating the subject with radiation based on the imaging condition that has been determined.

11. A radiographic image system, comprising the radiographic image analysis apparatus and the radiographic image capturing apparatus, wherein
the radiographic image analysis apparatus, comprising:
a hardware processor configured to:
acquire a plurality of frame images generated by performing dynamic imaging by irradiating a subject with radiation under an imaging condition corresponding to a combination of a plurality of types of dynamic analyses set in advance; and
execute, based on the plurality of frame images, the plurality of types of dynamic analyses included in the combination, and
the radiographic image capturing apparatus, comprising:
a hardware processor configured to:
acquire setting information on a combination of a plurality of types of dynamic analyses;
determine, based on the setting information, an imaging condition for dynamic imaging; and
generate a plurality of frame images by executing the dynamic imaging of a subject by irradiating the subject with radiation based on the imaging condition that has been determined.
